(19) United States
(12) Patent Application Publication
(10) Pub. No.: US 2016/0043316 A1
(43) Pub. Date: Feb. 11, 2016

(54) MONOAMINE MATERIAL FOR ORGANIC ELECTROLUMINESCENCE DEVICE AND ORGANIC ELECTROLUMINESCENCE DEVICE USING THE SAME

(71) Applicant: SAMSUNG DISPLAY CO., LTD., Yongin-City (KR)

(72) Inventors: Ichinori Takada, Yokohama (JP); Naoya Sakamoto, Yokohama (JP)

(21) Appl. No.: 14/706,137

(22) Filed: May 7, 2015

(30) Foreign Application Priority Data
Aug. 11, 2014 (JP) 2014-163347

(51) Int. Cl. H01L 51/00; C09K 11/06; C07D 333/50; C07C 211/61; C07F 7/08; C07D 409/04; C07D 307/91; C07D 333/76

(52) U.S. Cl. CPC H01L 51/006; C07D 307/91; C09K 11/06; H01L 51/0061; C07D 333/76; C07C 211/61; C07F 7/0812; H01L 51/0094; C07D 409/04; C07D 333/50; C09K 2211/1033; H01L 51/5206

(57) ABSTRACT

A compound for an organic electroluminescence device and an organic electroluminescence device, the compound being represented by the following Chemical Formula 1:

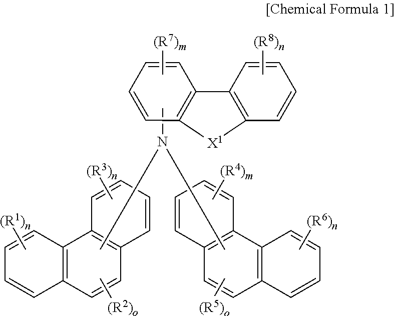

### CROSS-REFERENCE TO RELATED APPLICATION

Japanese Patent Application No. 2014-163347, filed on Aug. 11, 2014, in the Japanese Patent Office, and entitled: “Monoamine Material For Organic Electroluminescence Device and Organic Electroluminescence Device Using The Same,” is incorporated by reference herein in its entirety.

### BACKGROUND

1. Field

Embodiments relate to a monoamine material for an organic electroluminescence device and an organic electroluminescence device using the same.

2. Description of the Related Art

In recent years, organic electroluminescence displays (organic EL display) that are one type of image displays have been actively developed. Unlike a liquid crystal display and the like, the organic EL display is a self-luminescent display which recombines holes and electrons injected from an anode and a cathode in an emission layer to thus emit lights from a light-emitting material including an organic compound of the emission layer, thereby performing display.

An example of an organic electroluminescence device (hereinafter referred to as an organic EL device) may include an organic EL device which includes an anode, a hole transport layer disposed on the anode, an emission layer disposed on the hole transport layer, an electron transport layer disposed on the emission layer, and a cathode disposed on the electron transport layer. Holes injected from the anode are injected into the emission layer via the hole transport layer. Meanwhile, electrons are injected from the cathode, and then injected into the emission layer via the electron transport layer. The holes and the electrons injected into the emission layer are recombined to generate excitons within the emission layer. The organic EL device emits light by using light generated by deactivation of radiation of the excitons.

### SUMMARY

Embodiments are directed to a monoamine material for an organic electroluminescence device and an organic electroluminescence device using the same.

The embodiments may be realized by providing a compound for an organic electroluminescence device, the compound being represented by the following Chemical Formula 1:

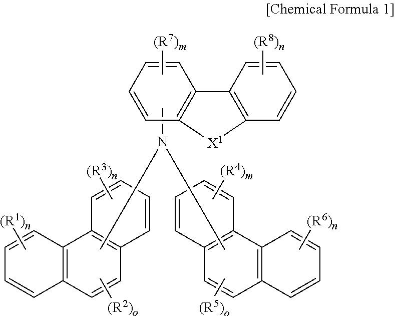

wherein, in Chemical Formula 1, X¹ is selected from O, S, R⁹—C—R¹⁰, or N—R¹¹, R¹ to R¹¹ are each independently an alkyl group having 1 to 10 carbon atoms, an aryl group having 6 to 30 ring carbon atoms, an alkyloxy group, an alkylthio group, a trialkylsilyl group, an aryloxy group having 6 to 30 ring carbon atoms, an arylthio group having 6 to 30 ring carbon atoms, a triarylsilyl group having 6 to 30 ring carbon atoms, an alkyldiarylsilyl group having 6 to 30 ring carbon atoms, a dialkylarylsilyl group having 6 to 30 ring carbon atoms, or a heteroaryl group having 2 to 30 ring carbon atoms, each n is independently an integer from about 0 to 4, each m is independently an integer from about 0 to 3, and each o is independently an integer from about 0 to 2.

In Chemical Formula 1, a phenanthryl group may be bound to a nitrogen atom at a position other than position 9 or position 10 of the phenanthryl group.

The embodiments may be realized by providing a compound for an organic electroluminescence device, the compound being represented by the following Chemical Formula 2:

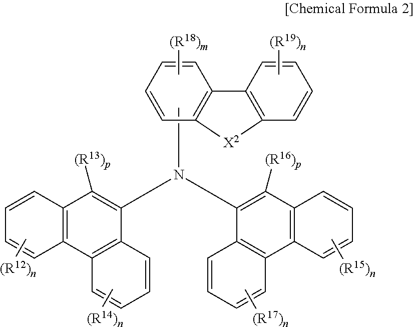

wherein, in Chemical Formula 2, X² is O or S, R¹² to R¹⁹ are each independently an alkyl group having 1 to 10 carbon atoms, an aryl group having 6 to 30 ring carbon atoms, an alkyloxy group, an alkylthio group, a trialkylsilyl group, an aryloxy group having 6 to 30 ring carbon atoms, an arylthio group having 6 to 30 ring carbon atoms, a triarylsilyl group having 6 to 30 ring carbon atoms, an alkyldiarylsilyl group having 6 to 30 ring carbon atoms, a dialkylarylsilyl group having 6 to 30 ring carbon atoms, or a heteroaryl group having 2 to 30 ring carbon atoms, each n is independently an integer from about 0 to 4, m is an integer from about 0 to 3, and each p is independently 0 or 1.

The embodiments may be realized by providing an organic electroluminescence device including an anode; an emission layer; and stacking layers between the anode and the emission layer, wherein at least one layer of the stacking layers between the anode and the emission layer includes a compound represented by the following Chemical Formula 1:

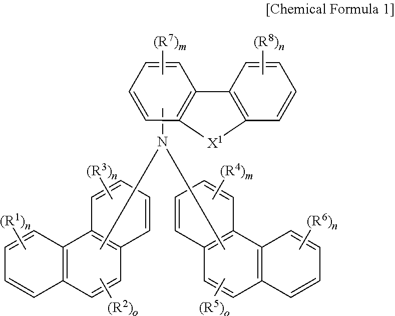

wherein, in Chemical Formula 1, X¹ is selected from O, S, R⁹—C—R¹⁰, or N—R¹¹, R¹ to R¹¹ are each independently an alkyl group having 1 to 10 carbon atoms, an aryl group having 6 to 30 ring carbon atoms, an alkyloxy group, an alkylthio group, a trialkylsilyl group, an aryloxy group having 6 to 30 ring carbon atoms, an arylthio group having 6 to 30 ring carbon atoms, a triarylsilyl group having 6 to 30 ring carbon atoms, an alkyldiarylsilyl group having 6 to 30 ring carbon atoms, a dialkylarylsilyl group having 6 to 30 ring carbon atoms, or a heteroaryl group having 2 to 30 ring carbon atoms, each n is independently an integer from about 0 to 4, each m is independently an integer from about 0 to 3, and each o is independently an integer from about 0 to 2.

The layer including the compound may be directly adjacent to the emission layer.

The compound may include one of the following Compounds 1 to 18:

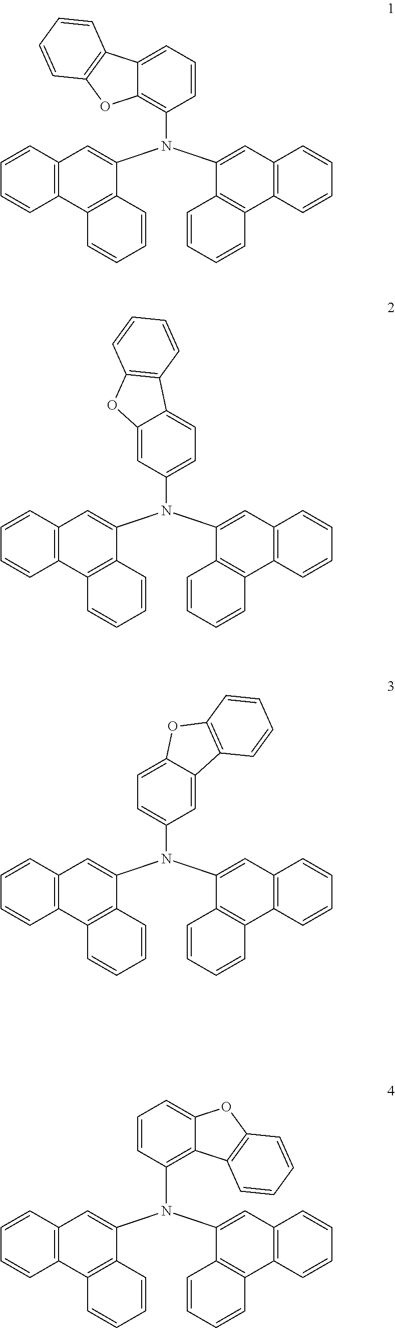 [SMILES: c1ccc2c(c1)cc(N(c1cc3ccccc3c3ccccc13)c1cccc3c1oc1ccccc13)c1ccccc12 ; c1ccc2c(c1)cc(N(c1ccc3c(c1)oc1ccccc13)c1cc3ccccc3c3ccccc13)c1ccccc12 ; c1ccc2c(c1)cc(N(c1ccc3oc4ccccc4c3c1)c1cc3ccccc3c3ccccc13)c1ccccc12 ; c1ccc2c(c1)cc(N(c1cc3ccccc3c3ccccc13)c1cccc3oc4ccccc4c13)c1ccccc12]

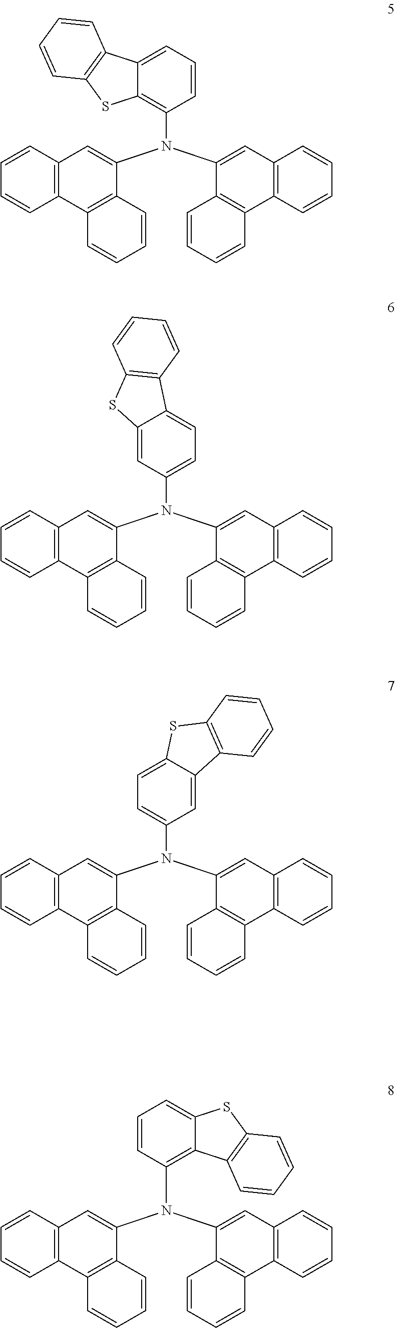 [SMILES: c1ccc2c(c1)cc(N(c1cc3ccccc3c3ccccc13)c1cccc3c1sc1ccccc13)c1ccccc12 ; c1ccc2c(c1)cc(N(c1ccc3c(c1)sc1ccccc13)c1cc3ccccc3c3ccccc13)c1ccccc12 ; c1ccc2c(c1)cc(N(c1ccc3sc4ccccc4c3c1)c1cc3ccccc3c3ccccc13)c1ccccc12 ; c1ccc2c(c1)cc(N(c1cc3ccccc3c3ccccc13)c1cccc3sc4ccccc4c13)c1ccccc12]

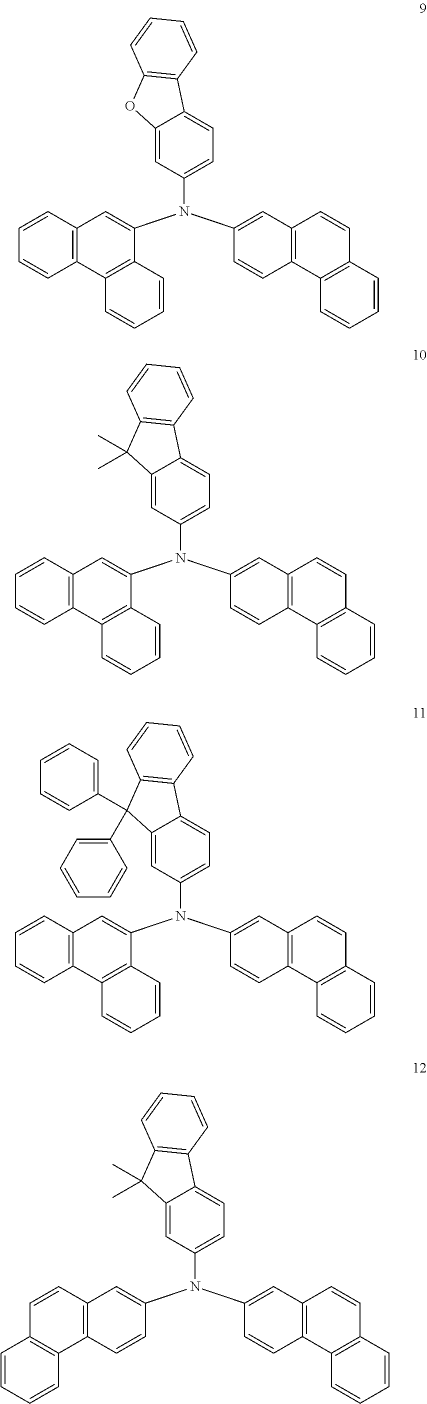

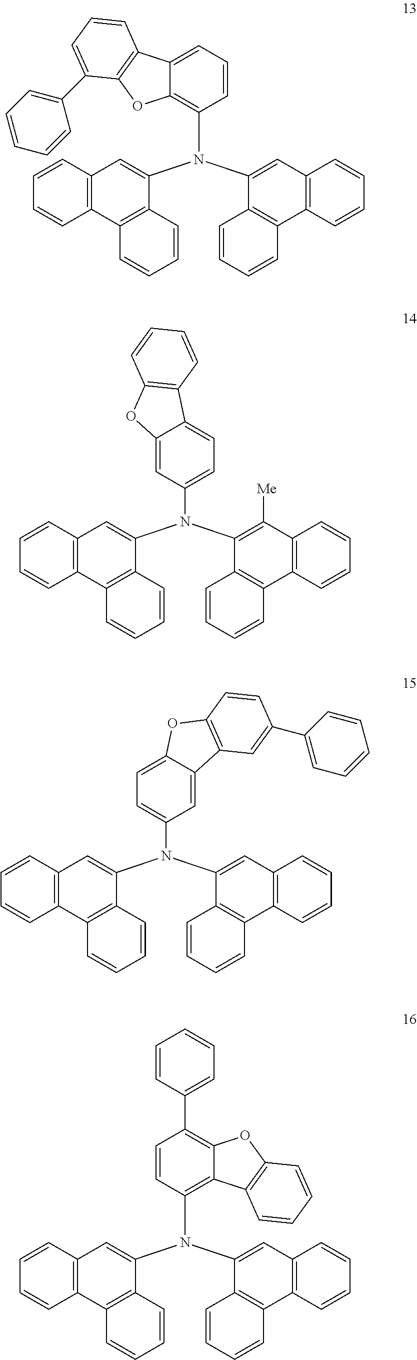 [SMILES: c1ccc(-c2cccc3c2oc2c(N(c4cc5ccccc5c5ccccc45)c4cc5ccccc5c5ccccc45)cccc23)cc1 ; Cc1c(N(c2ccc3c(c2)oc2ccccc23)c2cc3ccccc3c3ccccc23)c2ccccc2c2ccccc12 ; c1ccc(-c2ccc3oc4ccc(N(c5cc6ccccc6c6ccccc56)c5cc6ccccc6c6ccccc56)cc4c3c2)cc1 ; c1ccc(-c2ccc(N(c3cc4ccccc4c4ccccc34)c3cc4ccccc4c4ccccc34)c3c2oc2ccccc23)cc1]

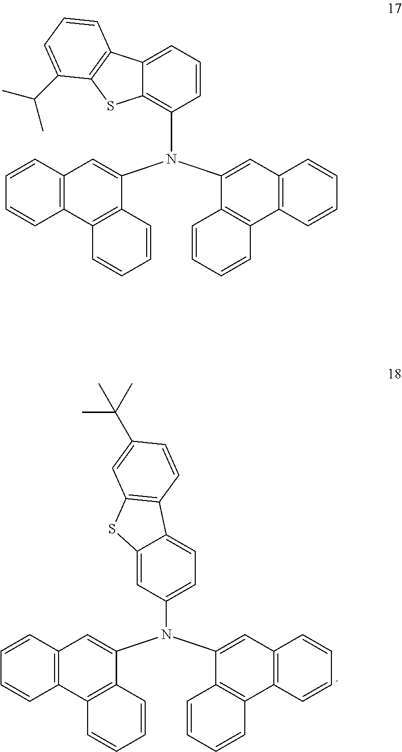 [SMILES: CC(C)c1cccc2c1sc1c(N(c3cc4ccccc4c4ccccc34)c3cc4ccccc4c4ccccc34)cccc12 ; CC(C)(C)c1ccc2c(c1)sc1cc(N(c3cc4ccccc4c4ccccc34)c3cc4ccccc4c4ccccc34)ccc12]

The compound may include one of the following Compounds 19 to 32:

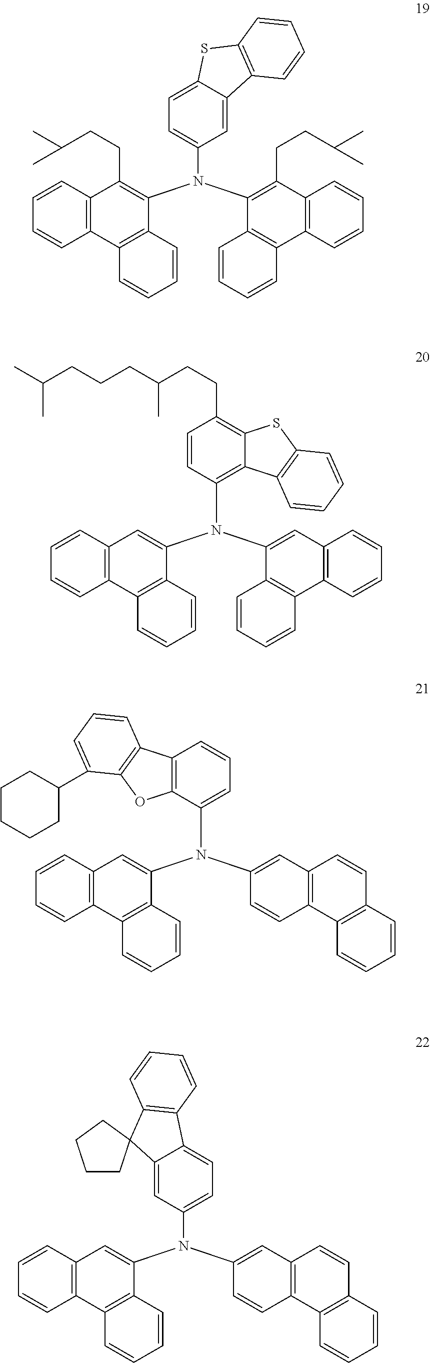

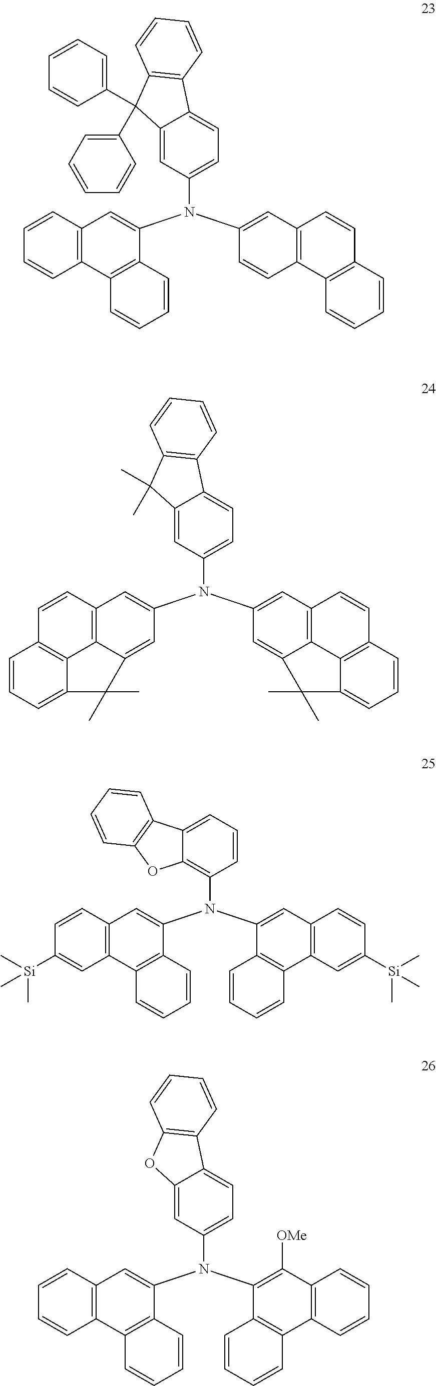

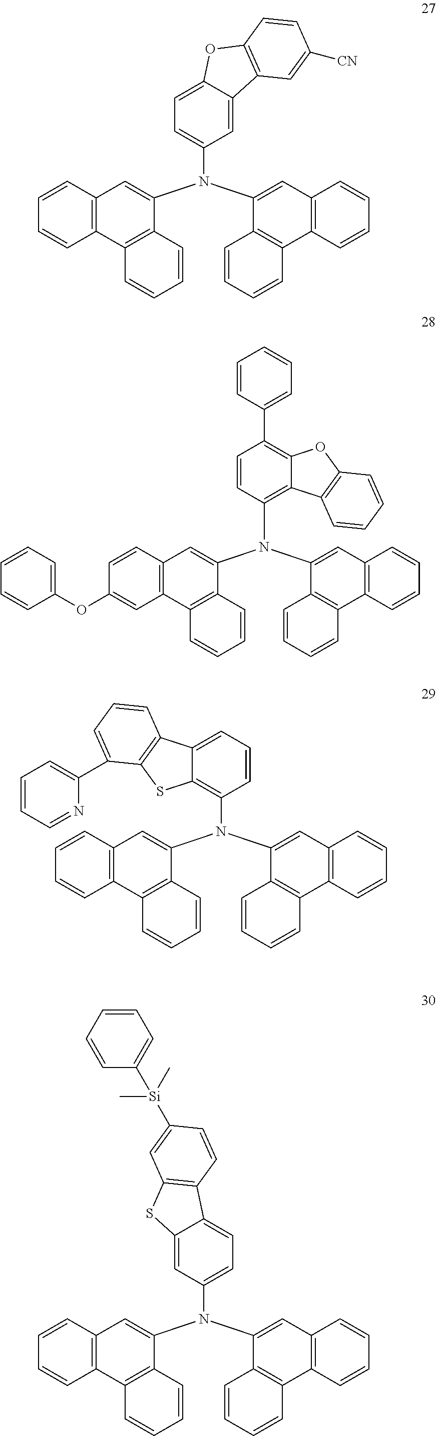 [SMILES: N#Cc1ccc2oc3ccc(N(c4cc5ccccc5c5ccccc45)c4cc5ccccc5c5ccccc45)cc3c2c1 ; c1ccc(Oc2ccc3cc(N(c4cc5ccccc5c5ccccc45)c4ccc(-c5ccccc5)c5oc6ccccc6c45)c4ccccc4c3c2)cc1 ; c1ccc(-c2cccc3c2sc2c(N(c4cc5ccccc5c5ccccc45)c4cc5ccccc5c5ccccc45)cccc23)nc1 ; C[Si](C)(c1ccccc1)c1ccc2c(c1)sc1cc(N(c3cc4ccccc4c4ccccc34)c3cc4ccccc4c4ccccc34)ccc12]

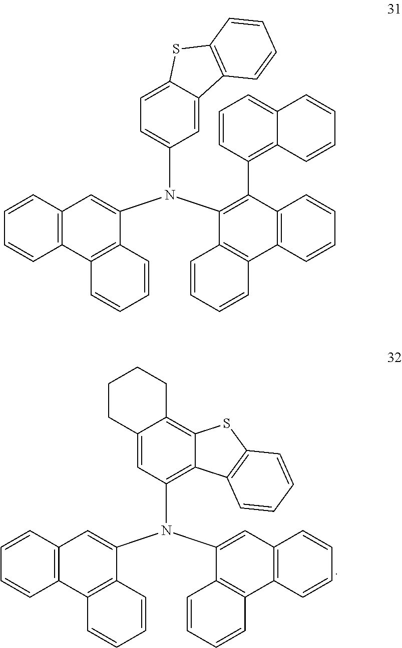 [SMILES: c1ccc2c(-c3c(N(c4ccc5sc6ccccc6c5c4)c4cc5ccccc5c5ccccc45)c4ccccc4c4ccccc34)cccc2c1 ; c1ccc2c(c1)cc(N(c1cc3ccccc3c3ccccc13)c1cc3c(c4sc5ccccc5c14)CCCC3)c1ccccc12]

The embodiments may be realized by providing an organic electroluminescence device including an anode; an emission layer; and stacking layers between the anode and the emission layer, wherein at least one layer of the stacking layers between the anode and the emission layer includes a compound represented by the following Chemical Formula 2:

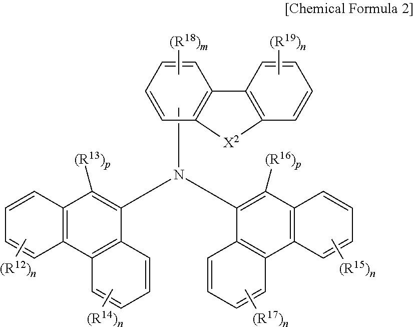

wherein, in Chemical Formula 2, X² is O or S, R¹² to R¹⁹ are each independently an alkyl group having 1 to 10 carbon atoms, an aryl group having 6 to 30 ring carbon atoms, an alkyloxy group, an alkylthio group, a trialkylsilyl group, an aryloxy group having 6 to 30 ring carbon atoms, an arylthio group having 6 to 30 ring carbon atoms, a triarylsilyl group having 6 to 30 ring carbon atoms, an alkyldiarylsilyl group having 6 to 30 ring carbon atoms, a dialkylarylsilyl group having 6 to 30 ring carbon atoms, or a heteroaryl group having 2 to 30 ring carbon atoms, each n is independently an integer from about 0 to 4, m is an integer from about 0 to 3, and each p is independently 0 or 1.

The layer including the compound may be directly adjacent to the emission layer.

### BRIEF DESCRIPTION OF THE DRAWINGS

Features will become apparent to those of ordinary skill in the art by describing in detail exemplary embodiments with reference to the attached drawings in which:

FIG. 1 illustrates a schematic diagram of an organic EL device according to an embodiment, and

FIG. 2 illustrates a schematic diagram of an organic EL device according to the embodiment.

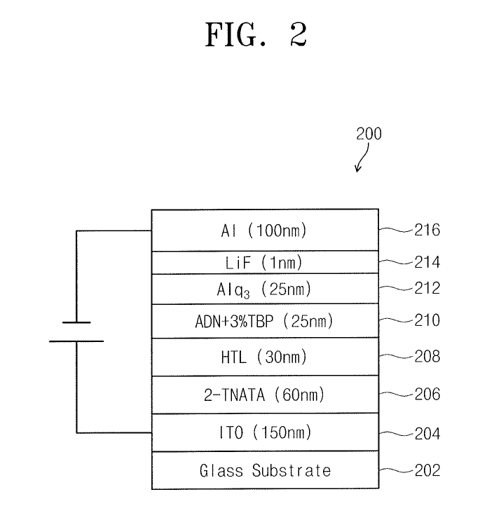

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. Like reference numerals refer to like elements throughout.

A compound, e.g., a monoamine material for an organic EL device, in which two phenanthrene portions are introduced onto a monoamine moiety, may be used in the organic EL device to realize high efficiency and a long life.

Hereinafter, a compound, e.g., a monoamine material, for an organic EL device and an organic EL device using the same according to exemplary embodiments will be described in detail with reference to the accompanying drawings.

The compound or material for an organic EL device according to an embodiment may be a monoamine-containing compound represented by the following Chemical Formula 1. For example, in the compound, two phenanthryl groups may be combined with a nitrogen atom (N) of an amine moiety.

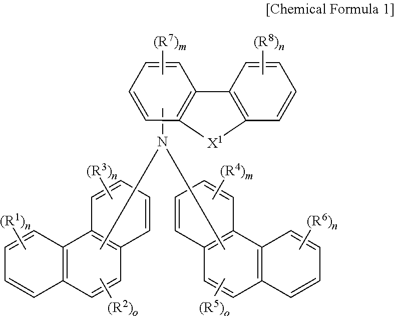

In Chemical Formula 1, X¹ may be, e.g., selected from O, S, R⁹—C—R¹⁰, or N—R¹¹, R¹ to R¹¹ may each independently be, e.g., an alkyl group having 1 to 10 carbon atoms, an aryl group, an alkyloxy group, an alkylthio group, a trialkylsilyl group, an aryloxy group, an arylthio group, a triarylsilyl group, an alkyldiarylsilyl group, a dialkylarylsilyl group (e.g., ring-containing groups may have 6 to 30 ring carbon atoms), or a heteroaryl group having 2 to 30 ring carbon atoms. Each n may independently be an integer from about 0 to 4, e.g., 0, 1, 2, 3, or 4, each m may independently be an integer from about 0 to 3, e.g., 0, 1, 2, or 3, and each o may independently be an integer from about 0 to 2, e.g., 0, 1, or, 2.

In an implementation, R¹ to R¹¹ may include, e.g., a methyl group, an ethyl group, a propyl group, an isopropyl group, a n-butyl group, a s-butyl group, an isobutyl group, a t-butyl group, a n-pentyl group, a n-hexyl group, a n-heptyl group, a n-octyl group, a hydroxymethyl group, a 1-hydroxyethyl group, a 2-hydroxyethyl group, a 2-hydroxyisobutyl group, a 1,2-dihydroxyethyl group, a 1,3-dihydroxyisopropyl group, a 2,3-dihydroxy-t-butyl group, a 1,2,3-trihydroxypropyl group, a chloromethyl group, a 1-chloroethyl group, a 2-chloroethyl group, a 2-chloroisobutyl group, a 1,2-dichloroethyl group, a 1,3-dichloroisopropyl group, a 2,3-dichloro-t-butyl group, a 1,2,3-trichloropropyl group, a bromomethyl group, a 1-bromoethyl group, a 2-bromoethyl group, a 2-bromoisobutyl group, a 1,2-dibromoethyl group, a 1,3-dibromoisopropyl group, a 2,3-dibromo-t-butyl group, a 1,2,3-tribromopropyl group, an iodomethyl group, a 1-iodoethyl group, a 2-iodoethyl group, a 2-iodoisobutyl group, a 1,2-diiodoethyl group, a 1,3-diiodoisopropyl group, a 2,3-diiodo-t-butyl group, a 1,2,3-triiodopropyl group, a cyanomethyl group, a 1-cyanoethyl group, a 2-cyanoethyl group, a 2-cyanoisobutyl group, a 1,2-dicyanoethyl group, a 1,3-dicyanoisopropyl group, a 2,3-dicyano-t-butyl group, a 1,2,3-tricyanopropyl group, a nitromethyl group, a 1-nitroethyl group, a 2-nitroethyl group, a 2-nitroisobutyl group, a 1,2-dinitroethyl group, a 1,3-dinitroisopropyl group, a 2,3-dinitro-t-butyl group, a 1,2,3-trinitropropyl group, a cyclopropyl group, a cyclobutyl group, a cyclopentyl group, a cyclohexyl group, a 4-methylcyclohexyl group, a 1-adamantyl group, a 2-adamantyl group, a 1-norbornyl group, a 2-norbornyl group, a phenyl group, a 1-naphthyl group, a 2-naphthyl group, a 1-anthryl group, a 2-anthryl group, a 9-anthryl group, a 1-phenanthryl group, a 2-phenanthryl group, a 3-phenanthryl group, a 4-phenanthryl group, a 9-phenanthryl group, a 1-naphthacenyl group, a 2-naphthacenyl group, a 9-naphthacenyl group, a 1-pyrenyl group, a 2-pyrenyl group, a 4-pyrenyl group, a 2-biphenylyl group, a 3-biphenylyl group, a 4-biphenylyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-tolyl group, a m-tolyl group, a p-tolyl group, a p-t-butylphenyl group, a p-(2-phenylpropyl)phenyl group, a 3-methyl-2-naphthyl group, a 4-methyl-1-naphthyl group, a 4-methyl-1-anthryl group, a 4′-methylbiphenylyl group, a 4-t-butyl-p-terphenyl-4-yl group, a fluoranthenyl group, a fluorenyl group, a 1-pyrrolyl group, a 2-pyrrolyl group, a 3-pyrrolyl group, a pyradinyl group, a 2-pyridinyl group, a 3-pyridinyl group, a 4-pyridinyl group, a 1-indolyl group, a 2-indolyl group, a 3-indolyl group, a 4-indolyl group, a 5-indolyl group, a 6-indolyl group, a 7-indolyl group, a 1-isoindolyl group, a 2-isoindolyl group, a 3-isoindolyl group, a 4-isoindolyl group, a 5-isoindolyl group, a 6-isoindolyl group, a 7-isoindolyl group, a 2-furyl group, a 3-furyl group, a 2-benzofuranyl group, a 3-benzofuranyl group, a 4-benzofuranyl group, a 5-benzofuranyl group, a 6-benzofuranyl group, a 7-benzofuranyl group, a 1-isobenzofuranyl group, a 3-isobenzofuranyl group, a 4-isobenzofuranyl group, a 5-isobenzofuranyl group, a 6-isobenzofuranyl group, a 7-isobenzofuranyl group, a quinolyl group, a 3-quinolyl group, a 4-quinolyl group, a 5-quinolyl group, a 6-quinolyl group, a 7-quinolyl group, a 8-quinolyl group, a 1-isoquinolyl group, a 3-isoquinolyl group, a 4-isoquinolyl group, a 5-isoquinolyl group, a 6-isoquinolyl group, a 7-isoquinolyl group, a 8-isoquinolyl group, a 2-quinoxalinyl group, a 5-quinoxalinyl group, a 6-quinoxalinyl group, a 1-carbazolyl group, a 2-carbazolyl group, a 3-carbazolyl group, a 4-carbazolyl group, a 9-carbazolyl group, a 1-dibenzofuranyl group, a 2-dibenzofuranyl group, a 3-dibenzofuranyl group, a 4-dibenzofuranyl group, a 1-dibenzothiazolyl group, a 2-dibenzothiazolyl group, a 3-dibenzothiazolyl group, a 4-dibenzothiazolyl group, a 1-phenanthridinyl group, a 2-phenanthridinyl group, a 3-phenanthridinyl group, a 4-phenanthridinyl group, a 6-phenanthridinyl group, a 7-phenanthridinyl group, a 8-phenanthridinyl group, a 9-phenanthridinyl group, a 10-phenanthridinyl group, a 1-acridinyl group, a 2-acridinyl group, a 3-acridinyl group, a 4-acridinyl group, a 9-acridinyl group, a 1,7-phenanthroline-2-yl group, a 1,7-phenanthroline-3-yl group, a 1,7-phenanthroline-4-yl group, a 1,7-phenanthroline-5-yl group, a 1,7-phenanthroline-6-yl group, a 1,7-phenanthroline-8-yl group, a 1,7-phenanthroline-9-yl group, a 1,7-phenanthroline-10-yl group, a 1,8-phenanthroline-2-yl group, a 1,8-phenanthroline-3-yl group, a 1,8-phenanthroline-4-yl group, a 1,8-phenanthroline-5-yl group, a 1,8-phenanthroline-6-yl group, a 1,8-phenanthroline-7-yl group, a 1,8-phenanthroline-9-yl group, a 1,8-phenanthroline-10-yl group, a 1,9-phenanthroline-2-yl group, a 1,9-phenanthroline-3-yl group, a 1,9-phenanthroline-4-yl group, a 1,9-phenanthroline-5-yl group, a 1,9-phenanthroline-6-yl group, a 1,9-phenanthroline-7-yl group, a 1,9-phenanthroline-8-yl group, a 1,9-phenanthroline-10-yl group, a 1,10-phenanthroline-2-yl group, a 1,10-phenanthroline-3-yl group, a 1,10-phenanthroline-4-yl group, a 1,10-phenanthroline-5-yl group, a 2,9-phenanthroline-1-yl group, a 2,9-phenanthroline-3-yl group, a 2,9-phenanthroline-4-yl group, a 2,9-phenanthroline-5-yl group, a 2,9-phenanthroline-6-yl group, a 2,9-phenanthroline-7-yl group, a 2,9-phenanthroline-8-yl group, a 2,9-phenanthroline-10-yl group, a 2,8-phenanthroline-1-yl group, a 2,8-phenanthroline-3-yl group, a 2,8-phenanthroline-4-yl group, a 2,8-phenanthroline-5-yl group, a 2,8-phenanthroline-6-yl group, a 2,8-phenanthroline-7-yl group, a 2,8-phenanthroline-9-yl group, a 2,8-phenanthroline-10-yl group, a 2,7-phenanthroline-1-yl group, a 2,7-phenanthroline-3-yl group, a 2,7-phenanthroline-4-yl group, a 2,7-phenanthroline-5-yl group, a 2,7-phenanthroline-6-yl group, a 2,7-phenanthroline-8-yl group, a 2,7-phenanthroline-9-yl group, a 2,7-phenanthroline-10-yl group, a 1-phenazinyl group, a 2-phenazinyl group, a 1-phenothiazinyl group, a 2-phenothiazinyl group, a 3-phenothiazinyl group, a 4-phenothiazinyl group, a 10-phenothiazinyl group, a 1-fenoxazinyl group, a 2-fenoxazinyl group, a 3-fenoxazinyl group, a 4-fenoxazinyl group, a 10-fenoxazinyl group, a 2-oxazolyl group, a 4-oxazolyl group, a 5-oxazolyl group, a 2-oxadiazolyl group, a 5-oxadiazolyl group, a 3-furazanyl group, a 2-thienyl group, a 3-thienyl group, a 2-methylpyrrol-1-yl group, a 2-methylpyrrol-3-yl group, a 2-methylpyrrol-4-yl group, a 2-methylpyrrol-5-yl group, a 3-methylpyrrol-1-yl group, a 3-methylpyrrol-2-yl group, a 3-methylpyrrol-4-yl group, a 3-methylpyrrol-5-yl group, a 2-t-butylpyrrol-4-yl group, a 3-(2-phenylpropyl)pyrrol-1-yl group, a 2-methyl-1-indolyl group, a 4-methyl-1-indolyl group, a 2-methyl-3-indolyl group, a 4-methyl-3-indolyl group, a 2-t-butyl1-indolyl group, a 4-t-butyl1-indolyl group, a 2-t-butyl3-indolyl group, a 4-t-butyl3-indolyl group, or the like.

In an implementation, in Chemical Formula 1, a plurality of adjacent ones of R¹-R¹¹ may be combined to form a saturated or unsaturated five to seven-membered ring.

In an implementation, in Chemical Formula 1, each of two phenanthryl groups may be independently combined or bound to a nitrogen atom (N) of the amine moiety at position 9 or position 10 of the phenanthryl groups.

For example, in Chemical Formula 1, in the case that two phenanthryl groups are each bound to the nitrogen atom (N) of the central amine at portion 9 or portion 10, the compound for an organic EL device according to an embodiment may be represented by the following Chemical Formula 2.

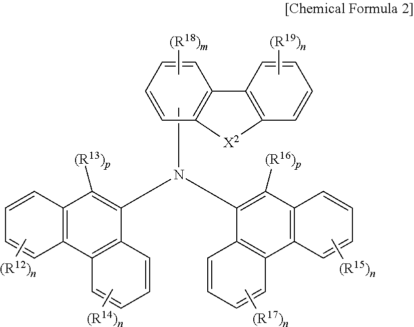

In Chemical Formula 2, X² may be, e.g., O or S, R¹² to R¹⁹ may each independently be, e.g., an alkyl group having 1 to 10 carbon atoms, an aryl group, an alkyloxy group, an alkylthio group, a trialkylsilyl group, an aryloxy group, an arylthio group, a triarylsilyl group, an alkyldiarylsilyl group, a dialkylarylsilyl group (e.g., ring-containing groups may have 6 to 30 ring carbon atoms), or a heteroaryl group having 2 to 30 ring carbon atoms. Each n may independently be an integer from about 0 to 4, m may be an integer from about 0 to 3, and each p may independently be 0 or 1.

In an implementation, R¹²-R¹⁹ may include, e.g., functional groups the same as or similar to those described with respect to R¹-R¹¹ in Chemical Formula 1. In an implementation, in Chemical Formula 2, a plurality of adjacent ones of R¹²-R¹⁹ may be combined or bound to form a saturated or unsaturated five to seven-membered ring.

In an implementation, in Chemical Formula 1, each of two phenanthryl groups may be bound to the nitrogen atom (N) of the amine moiety at a position other than position 9 or position 10 of the phenanthryl groups. In an implementation, in Chemical Formula 1, one of two phenanthryl groups may be bound to the nitrogen atom (N) of the amine at position 9 or position 10 of the phenanthryl group, and the other may be bound to the nitrogen atom (N) at a position other than position 9 or position 10 of the phenanthryl group. For example, two phenanthryl groups may be asymmetrically combined to the nitrogen atom (N) of the amine moiety of the compound.

In an implementation, the compound for an organic EL device according to an embodiment may be one of the following Compounds 1 to 32.

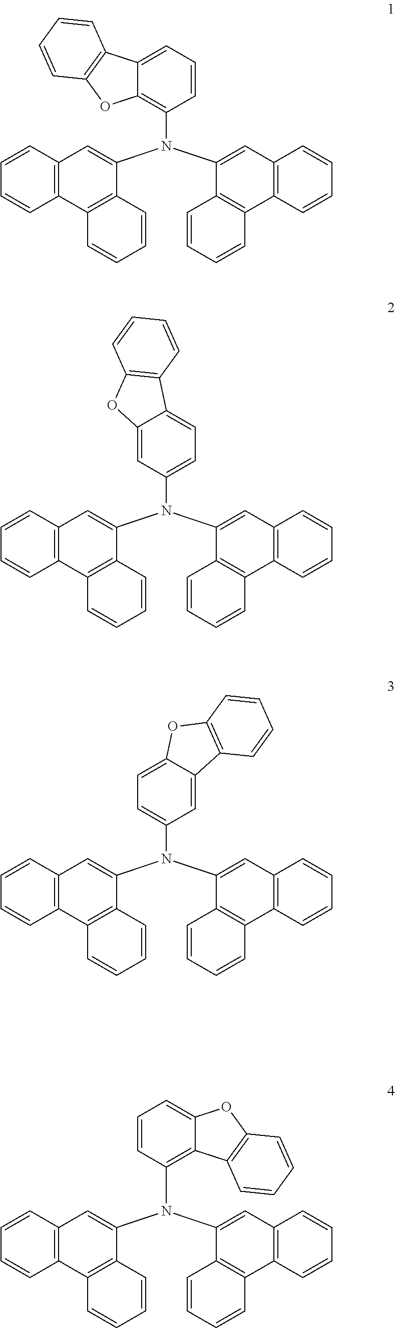 [SMILES: c1ccc2c(c1)cc(N(c1cc3ccccc3c3ccccc13)c1cccc3c1oc1ccccc13)c1ccccc12 ; c1ccc2c(c1)cc(N(c1ccc3c(c1)oc1ccccc13)c1cc3ccccc3c3ccccc13)c1ccccc12 ; c1ccc2c(c1)cc(N(c1ccc3oc4ccccc4c3c1)c1cc3ccccc3c3ccccc13)c1ccccc12 ; c1ccc2c(c1)cc(N(c1cc3ccccc3c3ccccc13)c1cccc3oc4ccccc4c13)c1ccccc12]

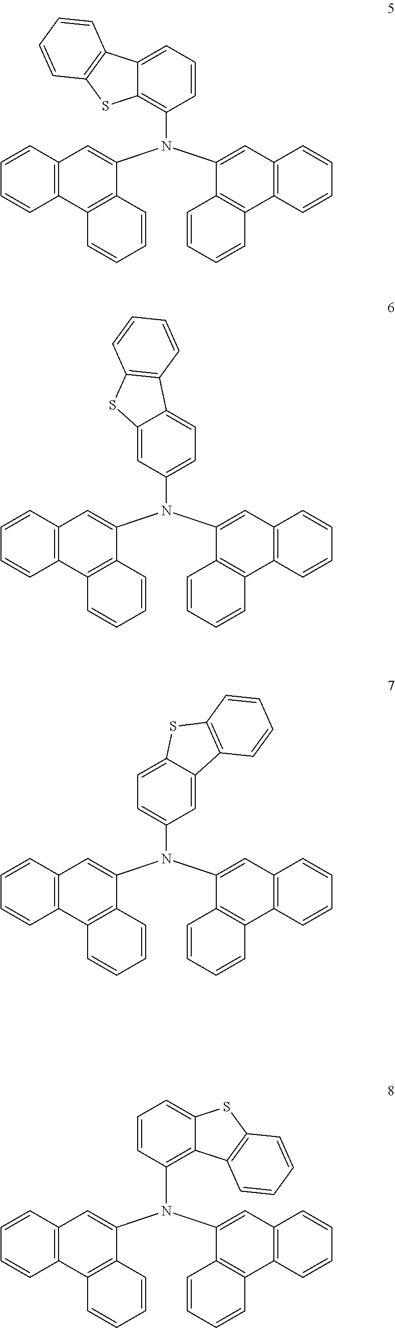 [SMILES: c1ccc2c(c1)cc(N(c1cc3ccccc3c3ccccc13)c1cccc3c1sc1ccccc13)c1ccccc12 ; c1ccc2c(c1)cc(N(c1ccc3c(c1)sc1ccccc13)c1cc3ccccc3c3ccccc13)c1ccccc12 ; c1ccc2c(c1)cc(N(c1ccc3sc4ccccc4c3c1)c1cc3ccccc3c3ccccc13)c1ccccc12 ; c1ccc2c(c1)cc(N(c1cc3ccccc3c3ccccc13)c1cccc3sc4ccccc4c13)c1ccccc12]

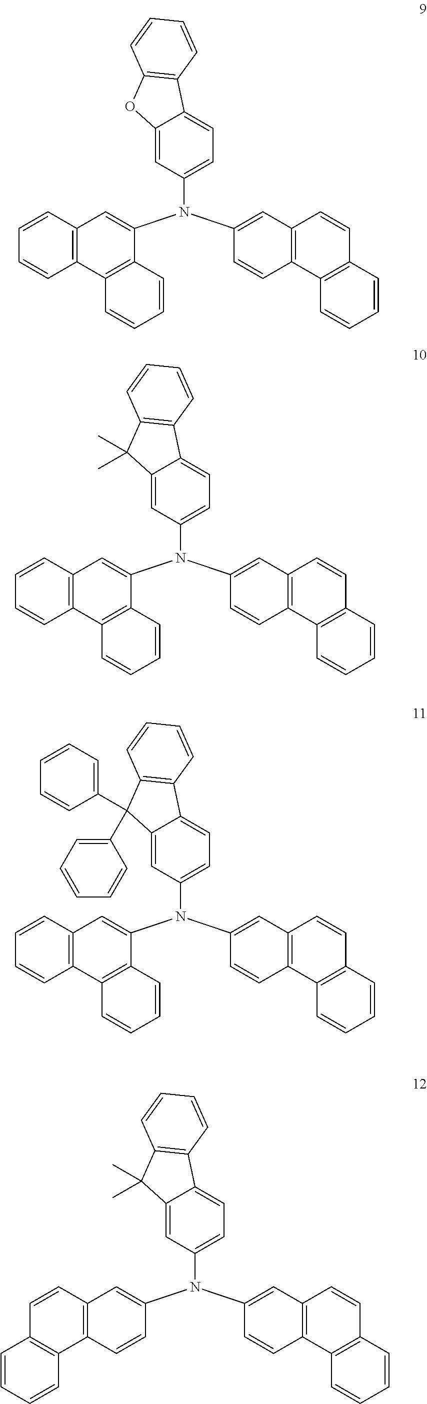

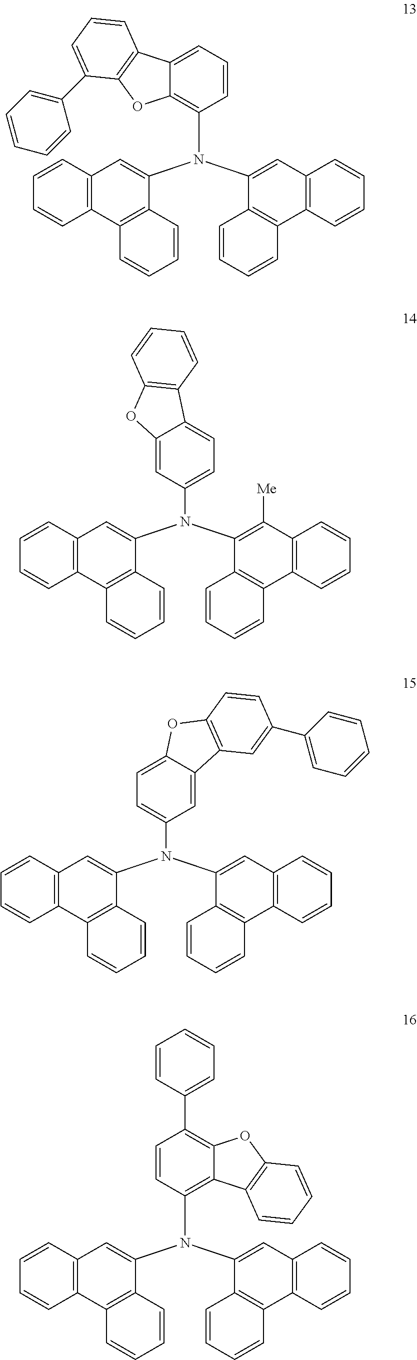 [SMILES: c1ccc(-c2cccc3c2oc2c(N(c4cc5ccccc5c5ccccc45)c4cc5ccccc5c5ccccc45)cccc23)cc1 ; Cc1c(N(c2ccc3c(c2)oc2ccccc23)c2cc3ccccc3c3ccccc23)c2ccccc2c2ccccc12 ; c1ccc(-c2ccc3oc4ccc(N(c5cc6ccccc6c6ccccc56)c5cc6ccccc6c6ccccc56)cc4c3c2)cc1 ; c1ccc(-c2ccc(N(c3cc4ccccc4c4ccccc34)c3cc4ccccc4c4ccccc34)c3c2oc2ccccc23)cc1]

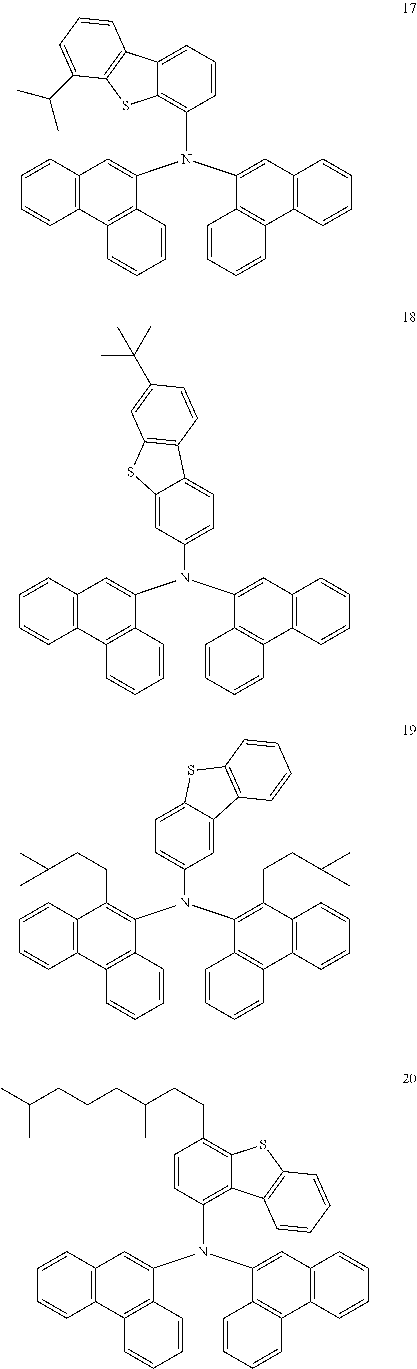

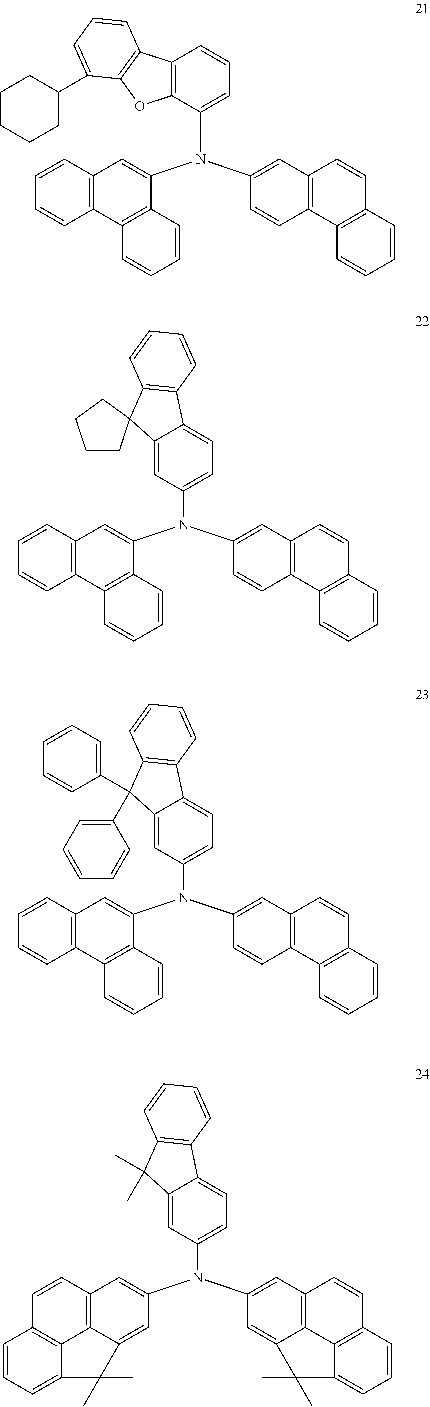 [SMILES: c1ccc2c(c1)ccc1cc(N(c3cc4ccccc4c4ccccc34)c3cccc4c3oc3c(C5CCCCC5)cccc34)ccc12 ; c1ccc2c(c1)-c1ccc(N(c3ccc4c(ccc5ccccc54)c3)c3cc4ccccc4c4ccccc34)cc1C21CCCC1 ; c1ccc(C2(c3ccccc3)c3ccccc3-c3ccc(N(c4ccc5c(ccc6ccccc65)c4)c4cc5ccccc5c5ccccc45)cc32)cc1 ; CC1(C)c2ccccc2-c2ccc(N(c3cc4c5c(ccc6cccc(c65)C4(C)C)c3)c3cc4c5c(ccc6cccc(c65)C4(C)C)c3)cc21]

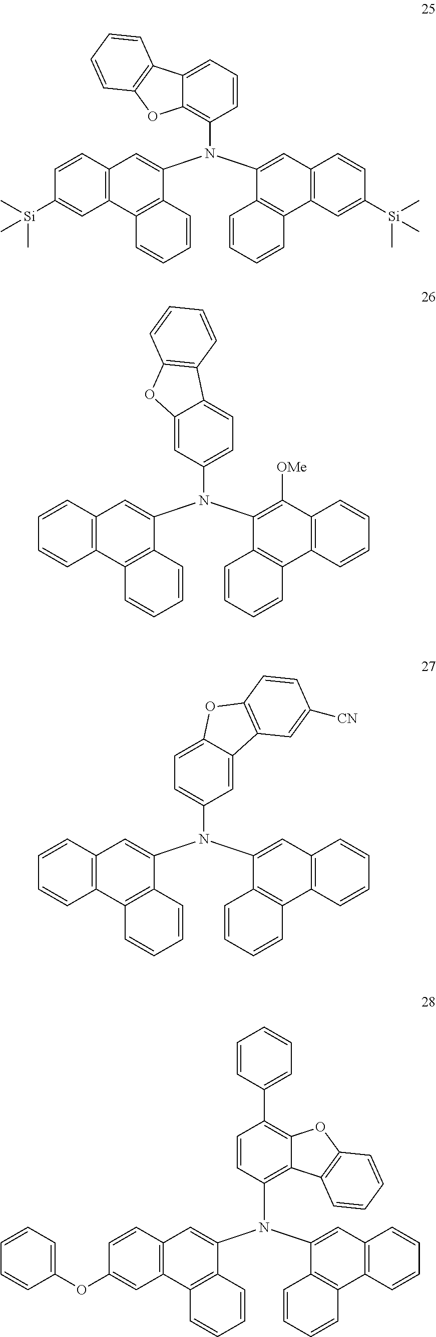

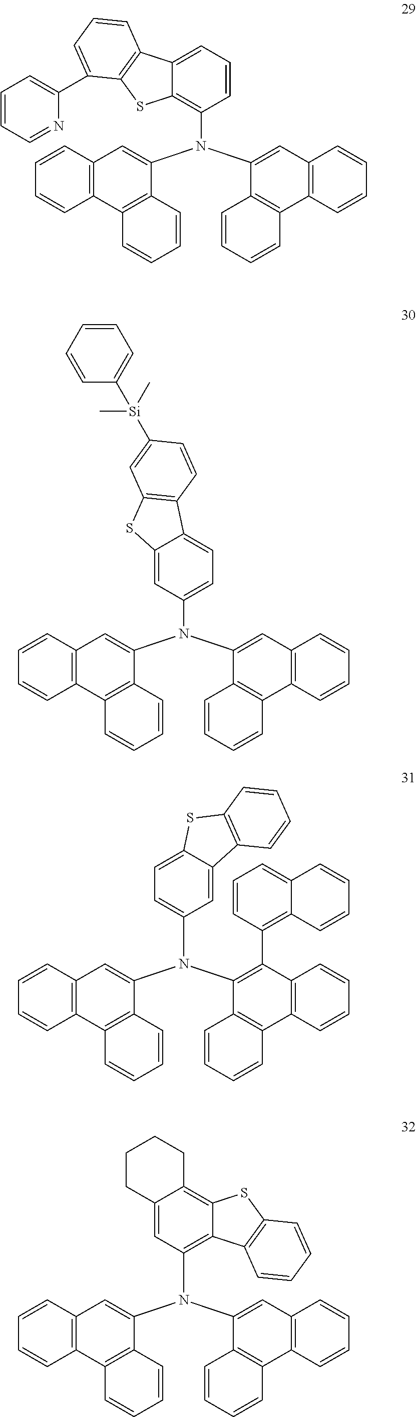

In the compound for an organic EL device according to an embodiment, hole transport properties and electron tolerance may be improved by introducing two phenanthrene portions to a central monoamine moiety. Accordingly, by using the compound for an organic EL device according to an embodiment as a material of a layer between the anode and the emission layer, high efficiency and a long life of the organic EL device may be achieved.

Further, in the compound for an organic EL device according to an embodiment, it is possible to hinder transferring of electrons by introducing two phenanthrene moieties onto a monoamine moiety. Transferring of the electrons not consumed in the emission layer but reaching the layer of the anode side may be hindered, and the compound for an organic EL device according to an embodiment may be used as the material of the layer between the anode and the emission layer to help suppress degradation due to the electrons of the layer of the anode side and contribute to the long life of the organic EL.

The compound for an organic EL device according to an embodiment may be used as the material of at least one layer of the layers between the anode and the emission layer in the organic EL device. For example, the compound be used as a material of a hole transport layer. In the case that the hole transport layer has a multilayered structure, in the multilayered structure, the compound used as the material of the layer reaching the emission layer, e.g., the layer adjacent to, directly adjacent to, or directly contacting the emission layer. By using the compound as the material of the layer reaching or, e.g., directly, adjacent to the emission layer of the layers between the anode and the emission layer, diffusion of electrons that not consumed in the emission layer to a layer at the anode side may be effectively suppressed to help reduce degradation of a layer at the anode side due to the electrons and may contribute to the long life of the organic EL.

(Organic EL Device)

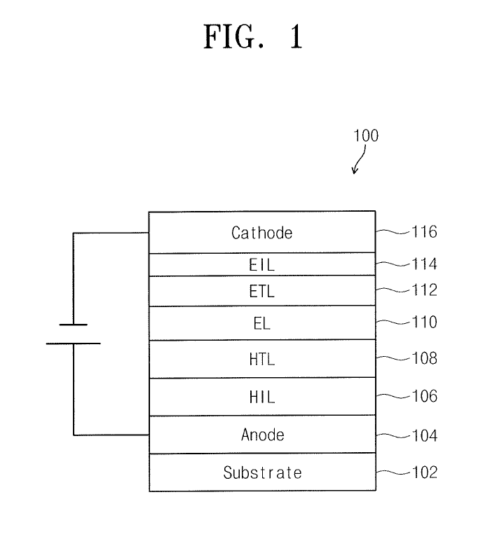

The organic EL device including the compound for an organic EL device according to an embodiment will be described. FIG. 1 illustrates a schematic diagram of an organic EL device 100 according to an embodiment. The organic EL device 100 may include, e.g., a substrate 102, an anode 104, a hole injection layer 106, a hole transport layer 108, an emission layer 110, an electron transport layer 112, an electron injection layer 114, and a cathode 116. In an implementation, the compound for an organic EL device according to an embodiment may be used or included in at least one layer of stacking layers between the emission layer and the anode.

For example, an embodiment including the compound in the hole transport layer 108 will be explained. The substrate 102, e.g., may be a transparent substrate, a semiconductor substrate formed of silicon or the like, or a flexible substrate such as a resin. The anode 104 may be disposed on the substrate 102 and formed by using, e.g., indium tin oxide (ITO), indium zinc oxide (IZO), or the like. The hole injection layer (HIL) 106 is disposed on the anode 104, and may include, for example, 4,4′,4″-tris (N-1-naphtyl-N-phenylamino)triphenylamine (1-TNATA), 4,4′,4″-tris(N-(2-naphthyl)-N-phenylamino)-triphenylamine (2-TNATA), 4,4-bis(N,N-di(3-tolyl)amino)-3,3-dimethylbiphenyl (HMTPD), or the like. The hole transport layer (HTL) 108 may be disposed on the hole injection layer 106 and may formed by using the compound according to an embodiment, e.g., the compound represented by Chemical Formula 1.

The emission layer (EL) 110 may be disposed on the hole transport layer 108, and formed, e.g., by doping 2,5,8,11-tetra-tert-butylperylene (TBPe) or the like on or into a host material including 9,10-di(2-naphthyl)anthracene (ADN) or the like. The electron transport layer (ETL) 112 is disposed on the emission layer 110 and formed from, e.g., a material including tris(8-hydroxyquinolinato)aluminum (Alq₃). The electron injection layer (EIL) 114 is disposed on the electron transport layer 112 and formed from, e.g., a material including lithium fluoride (LiF). The cathode (cathode) 116 is disposed on the electron injection layer 114 and formed from, e.g., a metal such as Al or a transparent material such as indium tin oxide (ITO) or indium zinc oxide (IZO). The thin layers may be formed by selecting an appropriate layer forming method according to a material, such as vacuum deposition, sputtering, and various coatings.

In the organic EL device 100 according to an embodiment, by using the compound described above, degradation of layers at the anode side of the device, e.g., due to the electrons, may be suppressed, and high efficiency and the long life of the organic EL device 100 may be realized.

In an implementation, in the organic EL device 100, the compound may be used as the material of the hole injection layer. As described above, the compound may be included in at least one layer of the stacking layers between the emission layer and the anode to help realize high efficiency and the long life of the organic EL device.

In an implementation, the compound may be applied even to an organic EL light-emitting apparatus of an active matrix using a TFT (thin film transistor). In an implementation, TBP (e.g., a fluorescent emission material) may be used as a dopant material of the emission layer, or a phosphorescent compound may be used as the material, e.g., dopant, of the emission layer.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

(Preparation Method)

The compound for an organic EL device may be synthesized by applying halogenides such as an aryl compound or a heteroaryl compound such as phenanthrene to the following Compound 33 or an arylamine compound having a similar structure by using a Pd catalyst or the like.

A compound for an organic EL device was synthesized by the following procedure.

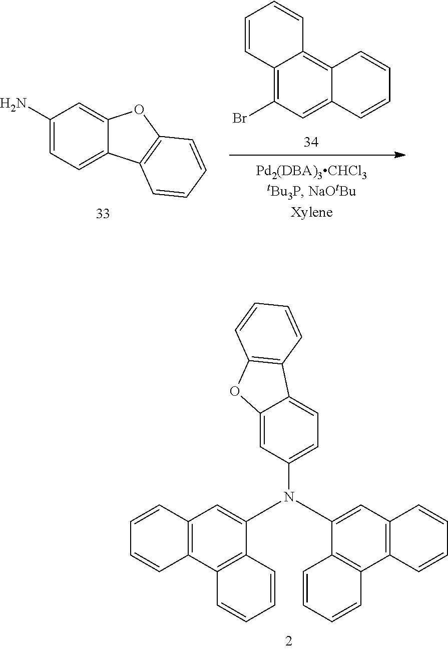 [SMILES: Nc1ccc2c(c1)oc1ccccc12 ; Brc1cc2ccccc2c2ccccc12 ; c1ccc2c(c1)cc(N(c1ccc3c(c1)oc1ccccc13)c1cc3ccccc3c3ccccc13)c1ccccc12]

(Synthesis of Compound 2)

A mixed solution of Compound 33 (dibenzofuranamine) (about 2.32 g, about 12.7 mmol), Compound 34 (bromophenanthrene) (about 6.68 g, about 25.6 mmol), sodium-tert-butoxide (NaOtBu) (about 3.73 g, about 38.0 mmol), a tris(dibenzylideneacetone)dipalladium(0)(chloroform) addition material (Pd₂(DBA)₃.CHCl₃) (about 656 mg, about 0.634 mmol), and anhydrous xylene (about 150 mL) was degasified, and a 1.6M-tri-tert-butylphosphine (tBU₃P) solution (about 475 μL, about 0.760 mmol) was added. The mixture was refluxed and heated for about 12 hours, cooled, and filtered. The filtrate was concentrated and purified by directly using column chromatography to obtain Compound 2 (about 4.62 g, about 68%) as a white powder.

(Identification Method of Compound 2)

Compound 2 was identified by detecting the molecular ion peak by FAB-MS measurement. The molecular weight of Compound 2 measured by FAB-MS measurement was about 535.

Another compound was synthesized by the following procedure.

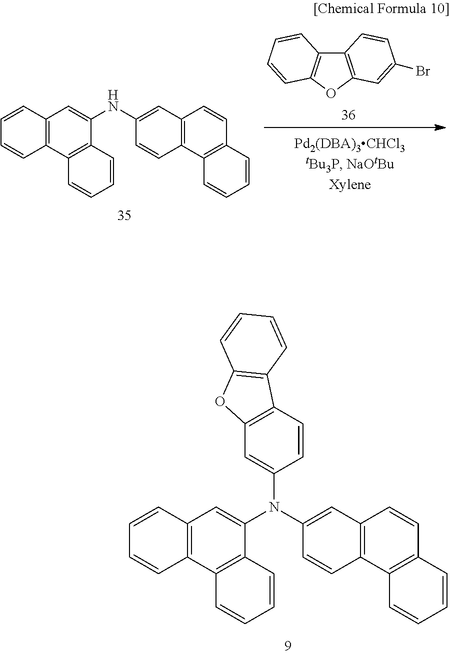 [SMILES: Brc1ccc2c(c1)oc1ccccc12 ; c1ccc2c(c1)ccc1cc(Nc3cc4ccccc4c4ccccc34)ccc12 ; c1ccc2c(c1)ccc1cc(N(c3ccc4c(c3)oc3ccccc34)c3cc4ccccc4c4ccccc34)ccc12]

(Synthesis of Compound 9)

A mixed solution of Compound 35 (diphenanthrylamine) (about 3.19 g, about 8.64 mmol), Compound 36 (bromodibenzofuran) (about 2.35 g, about 9.51 mmol), sodium-tert-butoxide (NaOtBu) (about 4.98 g, about 51.9 mmol), a tris(dibenzylideneacetone)dipalladium(0)(chloroform) addition material (Pd₂(DBA)₃.CHCl₃) (about 268 mg, about 0.260 mmol), and anhydrous xylene (about 100 mL) was degasified, and a 1.6M-tri-tert-butylphosphine (tBU₃P) solution (about 314 μL, about 0.520 mmol) was added. The mixture was refluxed and heated for about 10 hours, cooled, and filtered. The filtrate was concentrated and purified by directly using column chromatography to obtain Compound 9 (about 3.33 g, about 72%) as a white powder.

Compound 9 was identified by detecting the molecular ion peak by FAB-MS measurement. The molecular weight of Compound 9 measured by FAB-MS measurement was about 535.

The same procedure was performed to obtain Compound 11. Further, as Comparative Examples, the following Comparative Example Compounds c1 to c3 were prepared.

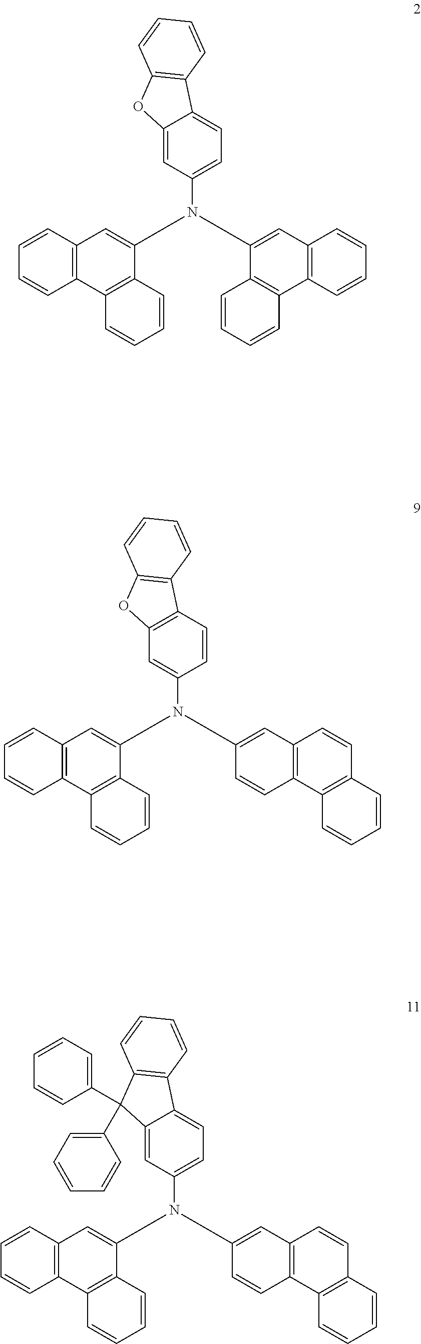 [SMILES: c1ccc2c(c1)cc(N(c1ccc3c(c1)oc1ccccc13)c1cc3ccccc3c3ccccc13)c1ccccc12 ; c1ccc2c(c1)ccc1cc(N(c3ccc4c(c3)oc3ccccc34)c3cc4ccccc4c4ccccc34)ccc12 ; c1ccc(C2(c3ccccc3)c3ccccc3-c3ccc(N(c4ccc5c(ccc6ccccc65)c4)c4cc5ccccc5c5ccccc45)cc32)cc1]

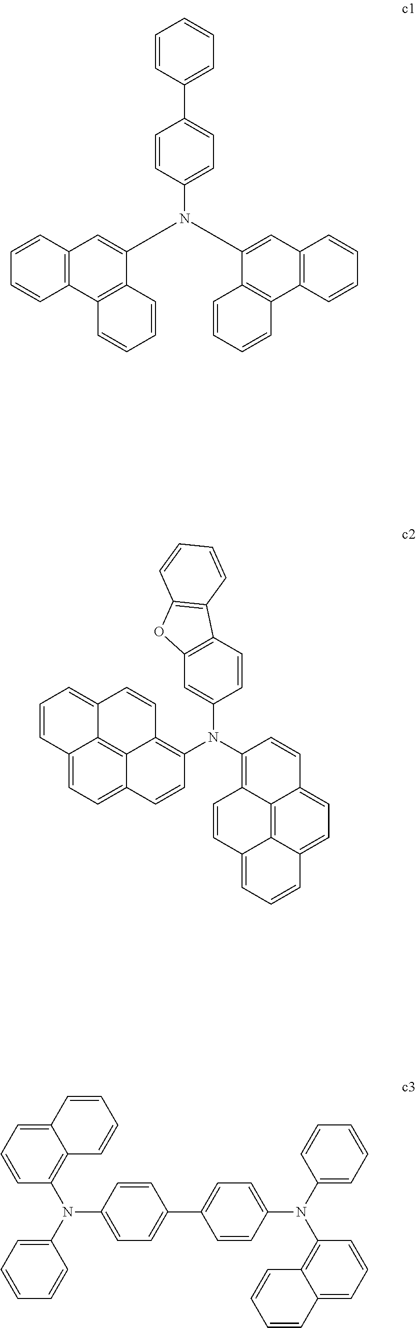 [SMILES: c1ccc(-c2ccc(N(c3cc4ccccc4c4ccccc34)c3cc4ccccc4c4ccccc34)cc2)cc1 ; c1cc2ccc3ccc(N(c4ccc5c(c4)oc4ccccc45)c4ccc5ccc6cccc7ccc4c5c67)c4ccc(c1)c2c34 ; c1ccc(N(c2ccc(-c3ccc(N(c4ccccc4)c4cccc5ccccc45)cc3)cc2)c2cccc3ccccc23)cc1]

Compounds 2, 9, and 11, and Comparative Example Compounds c1 to c3 were used as a hole transport material to form an organic EL device 200 like the organic EL device 100. A transparent glass substrate was used as the substrate 202, an anode 204 was formed using ITO having a thickness of about 150 nm, a hole injection layer 206 was formed using 2-TNATA having a thickness of about 60 nm, the hole transport layer 208 having a thickness of about 30 nm was formed using the compounds described above, an emission layer 210 having a thickness of about 25 nm was formed by doping about 3% of TBP in ADN, an electron transport layer 212 having a thickness of about 25 nm was formed using Alq₃, an electron injection layer 214 having a thickness of about 1 nm was formed using LiF, and a cathode 216 having a thickness of about 100 nm was formed using Al.

The driving voltage, emission efficiency, and the half-life of the manufactured organic EL device 200 were evaluated. Emission efficiency represents a value at about 10 mA/cm², and the half-life represents a time for which brightness is reduced by half from initial brightness of about 1,000 cd/m². The evaluation results are described in Table 1.

TABLE 1

| | | | Emission | Emission |
|---|---|---|---|---|
| | | Voltage | efficiency | life |
| | HTL | (V) | (cd/A) | (hour) |
| Example 1 | Compound 2 | 6.6 | 7.2 | 2,200 |
| Example 2 | Compound 9 | 6.6 | 7.2 | 2,400 |
| Example 3 | Compound 11 | 6.7 | 7.1 | 2,250 |
| Comparative | Comparative Example | 7.1 | 6.6 | 1,100 |
| Example 1 | Compound c1 | | | |
| Comparative | Comparative Example | 6.7 | 5.1 | 900 |
| Example 2 | Compound c2 | | | |
| Comparative | Comparative Example | 8.1 | 5.3 | 1,200 |
| Example 3 | Compound c3 | | | |

As apparent from Table 1, it may be in the EL device of Examples 1 to 3, as compared with Comparative Example 3 (including Comparative Example Compound c3 as a hole transport material), the driving voltage was reduced, and emission efficiency and a device life were improved. Further, it may be seen that in the case of comparison between Examples 1 to 3 and Comparative Example 1, the driving voltage was reduced, and emission efficiency and the device life were improved. Further, it may be seen that in the case of comparison between Example 1 and Comparative Example 2 (in which pyrene moieties were bound to a central amine moiety), emission efficiency and the device life were improved. Further, in the case of comparison between Examples 1 and 2, the life of Example 2 (using Compound 9 where one of two phenanthryl groups is combined to the nitrogen atom (N) of amine at position 9 and the other is combined to the nitrogen atom (N) at position 2, e.g., two phenanthryl moieties were asymmetrically combined to the nitrogen atom (N) of amine, was improved when compared to that of Example 1.

In the compound for an organic EL device according to an embodiment, two phenanthrene portions may be introduced onto the nitrogen atom of amine to help improve hole transport properties and electron tolerance and to help hinder transferring of electrons. Accordingly, as may be seen in Table 1, by using the compound as a material of at least one layer between the anode and the emission layer, high efficiency and the long life of the organic EL device may be achieved.

By way of summation and review, in the application of an organic EL device to a display apparatus, high efficiency and a long life of the organic device may be required. Normalization, stabilization, an increase in durability, and the like of a hole transport layer to help realize the high efficiency and a long life of the organic device may be considered.

Compounds, such as a hole transport material and an aromatic amine-based compound used in the hole transport layer may include, e.g., an amine-based compound. However, due to, e.g., low electron tolerance of the aromatic amine-based compound and the like, an organic EL device using the compound materials may not have a sufficient emission life. Further, a hole transport material may not be consumed in the emission layer and may lack a property of hindering transferring of electrons reaching a layer of an anode side, and degradation of the material (of the layer at the anode side) by the electrons may become a factor of reducing emission efficiency and a life of the organic EL device. Therefore, an organic EL device having higher efficiency and a longer emission life may be desirable. Emitting efficiency of the organic EL device in a blue emitting region may lower than that in a red emitting region and a green emitting region, and improvement of emission efficiency may be desirable.

The embodiments may provide a hole transport material for an organic electroluminescence device having high efficiency and a long life.

The embodiments may provide a material for an organic EL device having high efficiency and a long life, and an organic EL device using the same.

In the monoamine material for an organic EL device according to an embodiment, two phenanthrene moieties may be introduced onto an amine to help improve hole transport properties and electron tolerance and help hinder transferring of electrons, and in the organic EL device, may form a hole transport layer having high efficiency and a long life.

In the organic EL device according to an embodiment, the monoamine material for an organic EL device, in which two phenanthrene portions are introduced onto an amine group, may be included in any one of the stacking layers between the emission layer and the anode to help improve hole transport properties and electron tolerance, to help hinder transferring of electrons, and to help realize high efficiency and a long life.

According to an embodiment, it is possible to provide a monoamine material for an organic EL device having high efficiency and a long life, and an organic EL device using the same.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic electroluminescence device, the compound being represented by the following Chemical Formula 1: 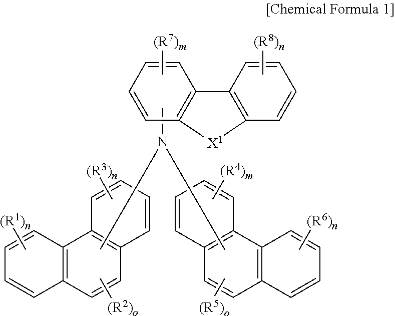
wherein, in Chemical Formula 1,
X¹ is selected from O, S, R⁹—C—R¹⁰, or N—R¹¹,
R¹ to R¹¹ are each independently an alkyl group having 1 to 10 carbon atoms, an aryl group having 6 to 30 ring carbon atoms, an alkyloxy group, an alkylthio group, a trialkylsilyl group, an aryloxy group having 6 to 30 ring carbon atoms, an arylthio group having 6 to 30 ring carbon atoms, a triarylsilyl group having 6 to 30 ring carbon atoms, an alkyldiarylsilyl group having 6 to 30 ring carbon atoms, a dialkylarylsilyl group having 6 to 30 ring carbon atoms, or a heteroaryl group having 2 to 30 ring carbon atoms,
each n is independently an integer from about 0 to 4,
each m is independently an integer from about 0 to 3, and
each o is independently an integer from about 0 to 2.

2. The compound as claimed in claim 1, wherein in Chemical Formula 1, a phenanthryl group is bound to a nitrogen atom at a position other than position 9 or position 10 of the phenanthryl group.

3. A compound for an organic electroluminescence device, the compound being represented by the following Chemical Formula 2: 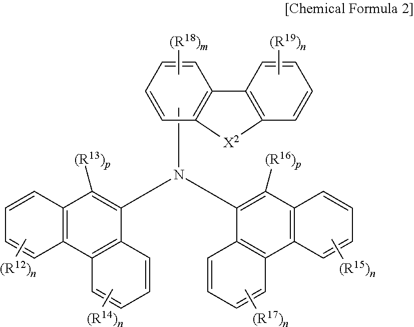
wherein, in Chemical Formula 2,
X² is O or S,
R¹² to R¹⁹ are each independently an alkyl group having 1 to 10 carbon atoms, an aryl group having 6 to 30 ring carbon atoms, an alkyloxy group, an alkylthio group, a trialkylsilyl group, an aryloxy group having 6 to 30 ring carbon atoms, an arylthio group having 6 to 30 ring carbon atoms, a triarylsilyl group having 6 to 30 ring carbon atoms, an alkyldiarylsilyl group having 6 to 30 ring carbon atoms, a dialkylarylsilyl group having 6 to 30 ring carbon atoms, or a heteroaryl group having 2 to 30 ring carbon atoms,
each n is independently an integer from about 0 to 4,
m is an integer from about 0 to 3, and
each p is 0 or 1.

4. An organic electroluminescence device, comprising:
an anode;
an emission layer; and
stacking layers between the anode and the emission layer,
wherein at least one layer of the stacking layers between the anode and the emission layer includes a compound represented by the following Chemical Formula 1: 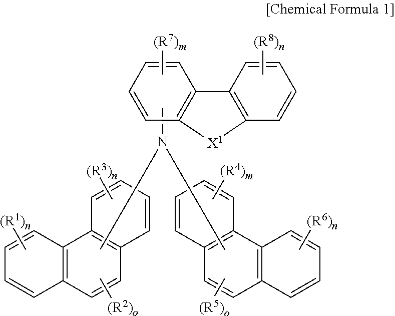
wherein, in Chemical Formula 1,
X¹ is selected from O, S, R⁹—C—R¹⁰, or N—R¹¹,
R¹ to R¹¹ are each independently an alkyl group having 1 to 10 carbon atoms, an aryl group having 6 to 30 ring carbon atoms, an alkyloxy group, an alkylthio group, a trialkylsilyl group, an aryloxy group having 6 to 30 ring carbon atoms, an arylthio group having 6 to 30 ring carbon atoms, a triarylsilyl group having 6 to 30 ring carbon atoms, an alkyldiarylsilyl group having 6 to 30 ring carbon atoms, a dialkylarylsilyl group having 6 to 30 ring carbon atoms, or a heteroaryl group having 2 to 30 ring carbon atoms,
each n is independently an integer from about 0 to 4,
each m is independently an integer from about 0 to 3, and
each o is independently an integer from about 0 to 2.

5. The organic electroluminescence device as claimed in claim 4, wherein the layer including the compound is directly adjacent to the emission layer.

6. The organic electroluminescence device as claimed in claim 4, wherein the compound includes one of the following Compounds 1 to 18:

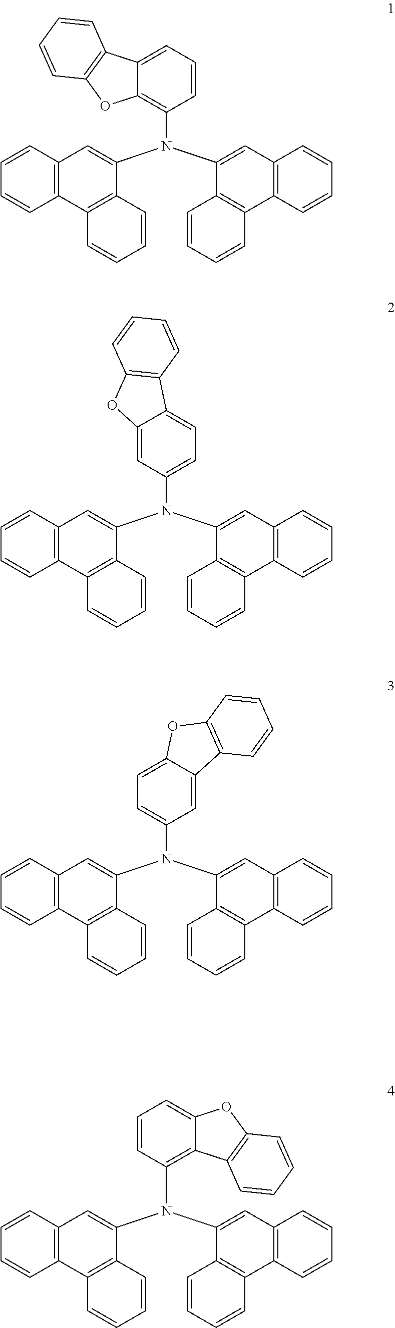 [SMILES: c1ccc2c(c1)cc(N(c1cc3ccccc3c3ccccc13)c1cccc3c1oc1ccccc13)c1ccccc12 ; c1ccc2c(c1)cc(N(c1ccc3c(c1)oc1ccccc13)c1cc3ccccc3c3ccccc13)c1ccccc12 ; c1ccc2c(c1)cc(N(c1ccc3oc4ccccc4c3c1)c1cc3ccccc3c3ccccc13)c1ccccc12 ; c1ccc2c(c1)cc(N(c1cc3ccccc3c3ccccc13)c1cccc3oc4ccccc4c13)c1ccccc12]

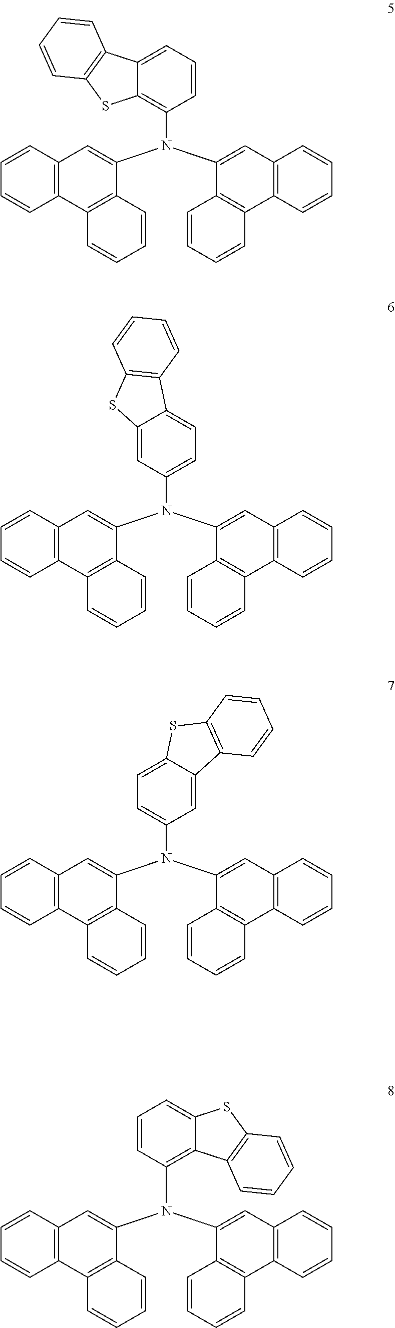 [SMILES: c1ccc2c(c1)cc(N(c1cc3ccccc3c3ccccc13)c1cccc3c1sc1ccccc13)c1ccccc12 ; c1ccc2c(c1)cc(N(c1ccc3c(c1)sc1ccccc13)c1cc3ccccc3c3ccccc13)c1ccccc12 ; c1ccc2c(c1)cc(N(c1ccc3sc4ccccc4c3c1)c1cc3ccccc3c3ccccc13)c1ccccc12 ; c1ccc2c(c1)cc(N(c1cc3ccccc3c3ccccc13)c1cccc3sc4ccccc4c13)c1ccccc12]

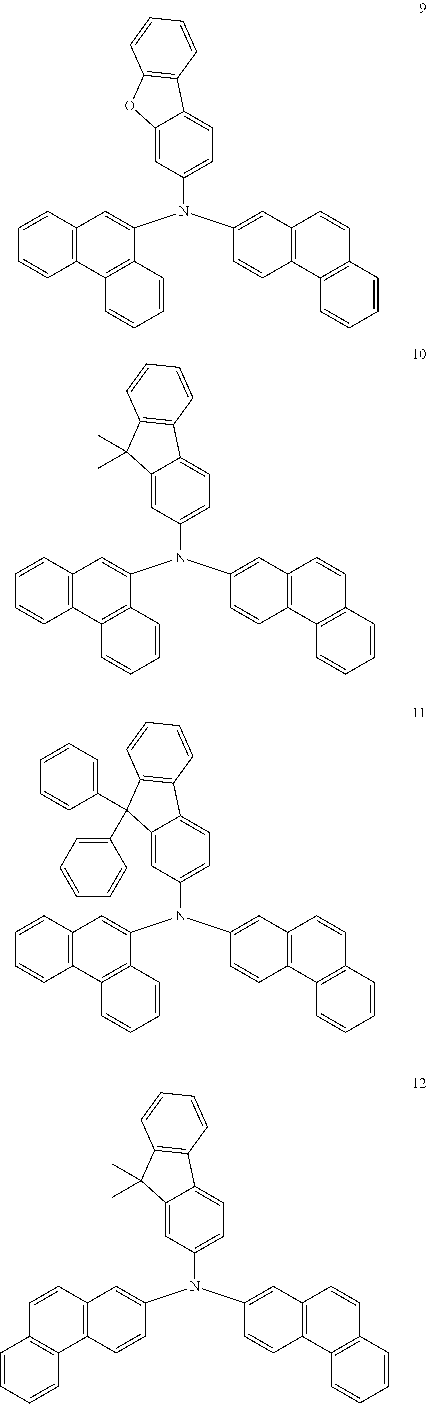

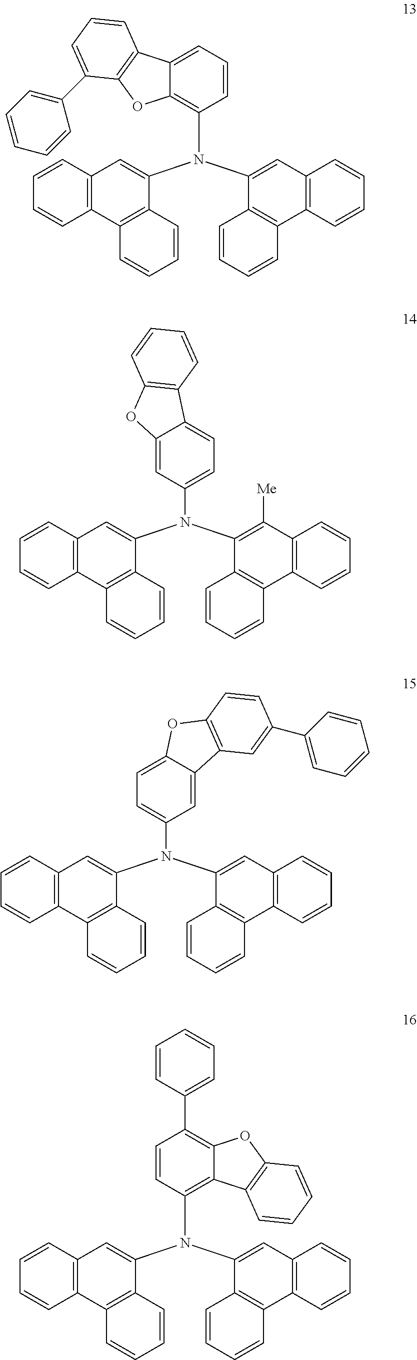 [SMILES: c1ccc(-c2cccc3c2oc2c(N(c4cc5ccccc5c5ccccc45)c4cc5ccccc5c5ccccc45)cccc23)cc1 ; Cc1c(N(c2ccc3c(c2)oc2ccccc23)c2cc3ccccc3c3ccccc23)c2ccccc2c2ccccc12 ; c1ccc(-c2ccc3oc4ccc(N(c5cc6ccccc6c6ccccc56)c5cc6ccccc6c6ccccc56)cc4c3c2)cc1 ; c1ccc(-c2ccc(N(c3cc4ccccc4c4ccccc34)c3cc4ccccc4c4ccccc34)c3c2oc2ccccc23)cc1]

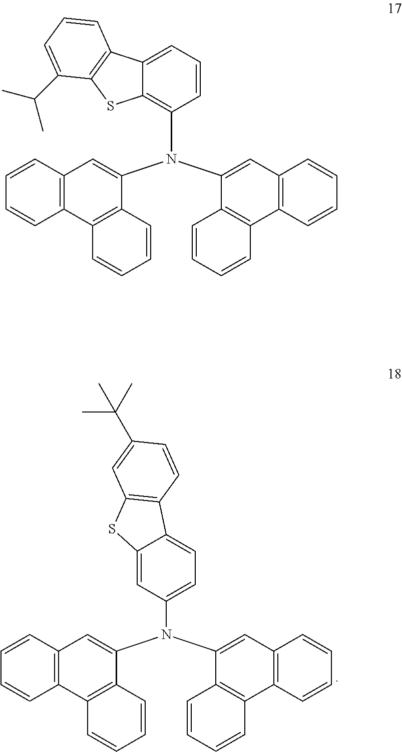 [SMILES: CC(C)c1cccc2c1sc1c(N(c3cc4ccccc4c4ccccc34)c3cc4ccccc4c4ccccc34)cccc12 ; CC(C)(C)c1ccc2c(c1)sc1cc(N(c3cc4ccccc4c4ccccc34)c3cc4ccccc4c4ccccc34)ccc12]

7. The organic electroluminescence device as claimed in claim 4, wherein the compound includes one of the following Compounds 19 to 32:

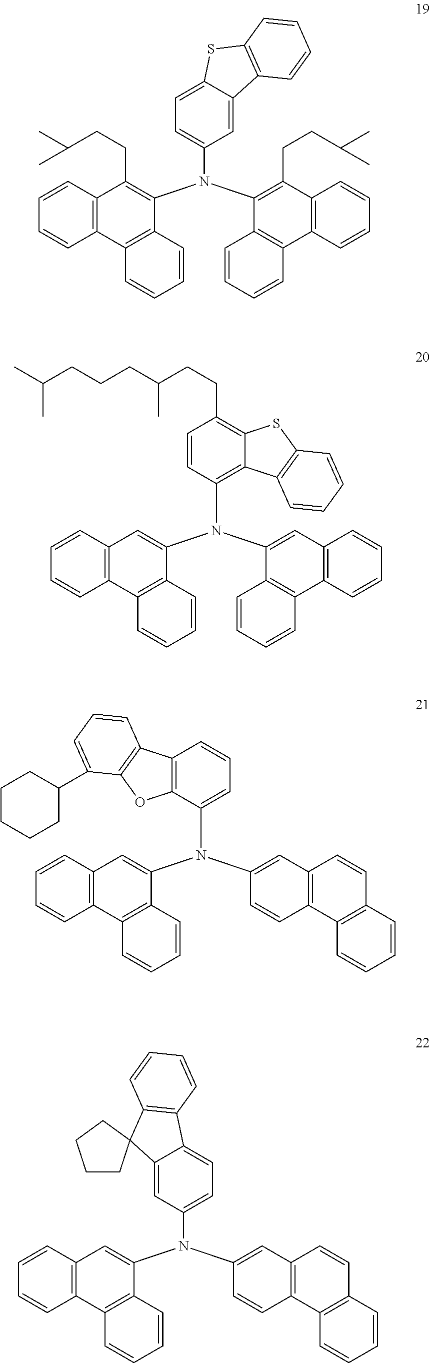

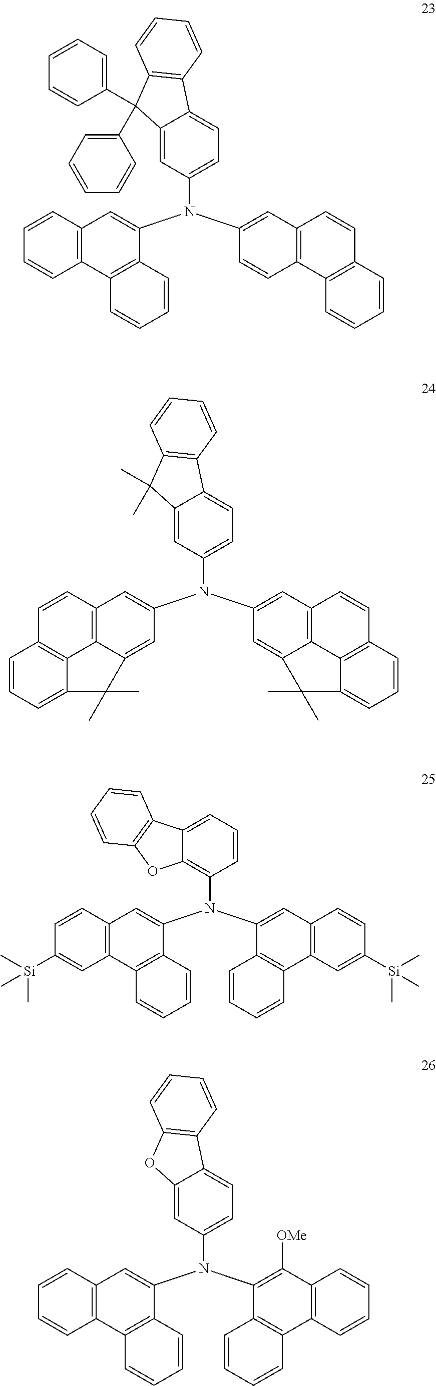

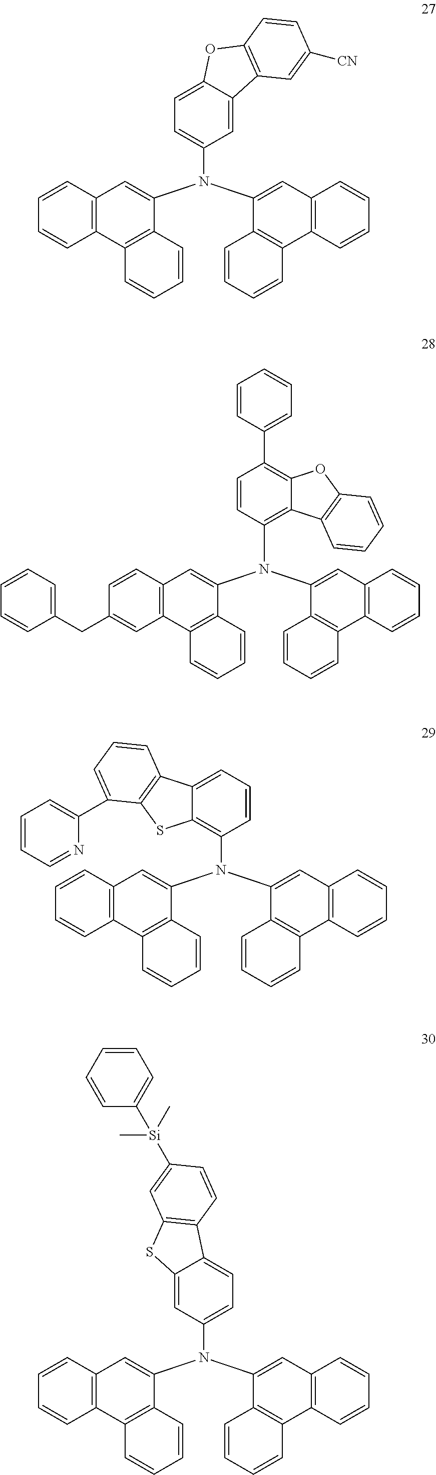 [SMILES: N#Cc1ccc2oc3ccc(N(c4cc5ccccc5c5ccccc45)c4cc5ccccc5c5ccccc45)cc3c2c1 ; c1ccc(Cc2ccc3cc(N(c4cc5ccccc5c5ccccc45)c4ccc(-c5ccccc5)c5oc6ccccc6c45)c4ccccc4c3c2)cc1 ; c1ccc(-c2cccc3c2sc2c(N(c4cc5ccccc5c5ccccc45)c4cc5ccccc5c5ccccc45)cccc23)nc1 ; C[Si](C)(c1ccccc1)c1ccc2c(c1)sc1cc(N(c3cc4ccccc4c4ccccc34)c3cc4ccccc4c4ccccc34)ccc12]

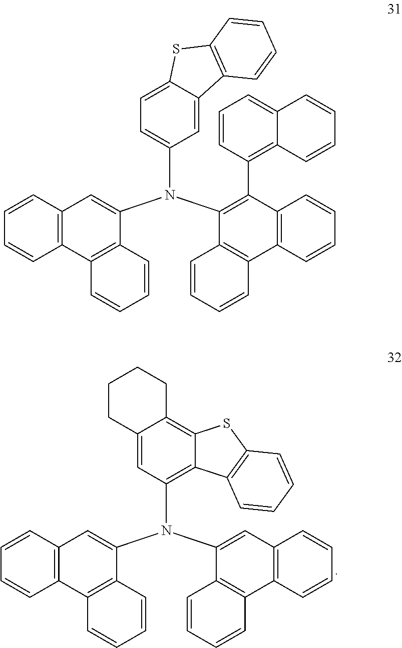 [SMILES: c1ccc2c(-c3c(N(c4ccc5sc6ccccc6c5c4)c4cc5ccccc5c5ccccc45)c4ccccc4c4ccccc34)cccc2c1 ; c1ccc2c(c1)cc(N(c1cc3ccccc3c3ccccc13)c1cc3c(c4sc5ccccc5c14)CCCC3)c1ccccc12]

8. An organic electroluminescence device, comprising:
an anode;
an emission layer; and
stacking layers between the anode and the emission layer,
wherein at least one layer of the stacking layers between the anode and the emission layer includes a compound represented by the following Chemical Formula 2: 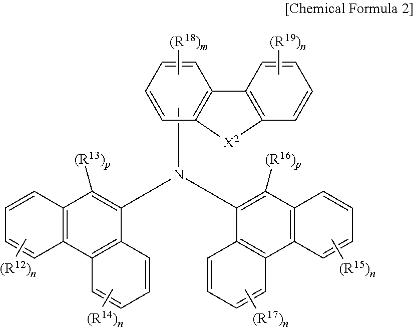
wherein, in Chemical Formula 2,
X² is O or S,
R¹² to R¹⁹ are each independently an alkyl group having 1 to 10 carbon atoms, an aryl group having 6 to 30 ring carbon atoms, an alkyloxy group, an alkylthio group, a trialkylsilyl group, an aryloxy group having 6 to 30 ring carbon atoms, an arylthio group having 6 to 30 ring carbon atoms, a triarylsilyl group having 6 to 30 ring carbon atoms, an alkyldiarylsilyl group having 6 to 30 ring carbon atoms, a dialkylarylsilyl group having 6 to 30 ring carbon atoms, or a heteroaryl group having 2 to 30 ring carbon atoms,
each n is independently an integer from about 0 to 4,
m is an integer from about 0 to 3, and
each p is independently 0 or 1.

9. The organic electroluminescence device as claimed in claim 8, wherein the layer including the compound is directly adjacent to the emission layer.
